(19) United States
(12) Patent Application Publication
(10) Pub. No.: US 2007/0134157 A1
(43) Pub. Date: Jun. 14, 2007

(54) Biological assay

(76) Inventor: Christopher P. Regan, North Wales, PA (US)

Correspondence Address: MERCK AND CO., INC, P O BOX 2000, RAHWAY, NJ, 07065-0907, US

(21) Appl. No.: 10/588,929

(22) PCT Filed: Feb. 22, 2005
(86) PCT No.: PCT/US05/05471

§ 371 Date: Aug. 9, 2006

Related U.S. Application Data
(60) Provisional application No. 60/547,875, filed on Feb. 26, 2004.

(51) Int. Cl. A61K 49/00

(52) U.S. Cl. 424/9.2

(57) ABSTRACT

The invention is a method for determining an in vivo cardiac electrophysiology profile of a compound affecting one or more cardiac ion channels which comprises administering the compound to a rat, and simultaneously measuring one or more periods selected from the group consisting of an atrial refractory period, a ventricular refractory period, and an AV nodal refractory period, and one or more intervals selected from an electrocardiogram interval and a cardiac electrogram conduction interval.

### BACKGROUND OF THE INVENTION

Methods for determining cardiac refractory periods in vivo in rats (examples: Hayes, et al. 2002. Pharm Res. 46(1): 19-29 and Saito, et al. 2002. Circ J. 66: 97-103) have been described. These methods involved direct insertion of pacing/recording wires into the ventricle either through the chest wall or via thoracotomy. Using these methods, it is possible to measure ventricular refractory periods. These methods do not provide a means to measure atrial and ventricular refractory periods as well as cardiac conduction parameters simultaneously, and accordingly, do not provide a detailed analysis of cardiac conduction and electrophysiologic parameters. Analyses of cardiac conduction and atrial/ventricular refractory periods in mice have been described (Gehrmann and Berul. 2000. J Cardiovasc Electrophysiol. 11: 354-368 and Rakhit et al. 2001. J Cardiovasc Electrophysiol. 12: 1295-1301).

Previous methods used as initial screens of novel compounds for cardiac electrophysiology activity relied heavily on isolated cardiac tissue or intact, isolated, perfused hearts. While these methods can determine the compound-dependent effects on certain cardiac electrophysiology parameters, they provide no integrative data on compound dependent effects in vivo, and cannot provide information on in vivo potency.

The present invention provides a method to assay the comprehensive in vivo cardiac electrophysiology profile of novel compounds in intact rats.

### SUMMARY OF THE INVENTION

The invention is a method for determining an in vivo cardiac electrophysiology profile of a compound affecting one or more cardiac ion channels which comprises administering the compound to a rat, and simultaneously measuring one or more periods selected from the group consisting of an atrial refractory period, a ventricular refractory period, and an AV nodal refractory period, and one or more intervals selected from an electrocardiogram interval and a cardiac electrogram conduction interval.

The method is useful for the development of novel antiarrhythmics targeting various cardiac ion channels, specifically, but not limited to the development of Kv1.5 antagonists, and for assessing whether novel compounds, targeted against a broad range of receptors, channels, and enzymes, have significant off-target activity, specifically, but not limited to IKr blocking activity, that affects cardiac electrophysiology. The method is useful for developing treatments for cardiac arrhythmias or cardiac conduction abnormalities.

### DETAILED DESCRIPTION OF THE INVENTION AND PREFERRED EMBODIMENTS

The present invention is a method to assay the comprehensive in vivo cardiac electrophysiology profile of novel compounds in intact rats. Information acquired from using this technique is similar to complete invasive cardiac electrophysiologic methods traditionally used in larger animals or humans. This method allows for complete electrophysiology profile of compounds to be generated faster, and with significantly lower cost and compound requirement compared to traditional large animal electrophysiology methods, and thus can serve as an initial screen of cardiac electrophysiology activity of novel compounds.

In one embodiment, the method involves
1) cannulating the left femoral artery of the rat with a first catheter,
2) cannulating the left femoral vein of the rat with a second catheter and the right femoral vein of the rat with a third catheter,
3) introducing a first recording and stimulating catheter into the right jugular vein of the rat, and advancing the first recording and stimulating catheter into or near the right atrium of the rat,
4) advancing a second recording and stimulating catheter down the right common carotid⁻ of the rat into the left ventricle of the rat,
5) placing needle electrodes subcutaneously at the right axillary and left inguinal areas of the rat,
6) administering the test compound either continuously or intermittently intravenously, and
7) determining one or more intervals selected from the group consisting of an electrocardiogram interval and a cardiac electrogram conduction interval and one or more periods selected from the group consisting of an atrial refractory period, a ventricular refractory period, and an AV nodal refractory period.

Step 3 is conducted in order to make one or more measurements selected from the group consisting of an atrial electrogram, atrial refractoriness, and AV nodal refractoriness, and/or to pace the heart of the rat.

Step 4 is conducted to record one or more electrograms selected from an atrial electrogram, a ventricular electrogram, and a His bundle electrogram, and/or pace the heart from the left ventricle.

Step 5 is conducted to record lead II electrocardiograms.

Step 6 may also be conducted by administering the compound by a means other than intravenous, e.g., transdermal, oral, etc.

In another embodiment, the test compound is a Kv1.5 antagonist.

In another embodiment, the cardiac ion channel is the Kv1.5 potassium ion channel.

In another embodiment, the test compound is a sodium channel antagonist.

In another embodiment, the test compound is a calcium channel antagonist.

In another embodiment, the test compound is an ERG potassium channel inhibitor. HERG is the potassium channel protein ether-a-go-go gene.

In another embodiment, the test compound is a cardiac refractoriness modifier or a cardiac conduction modifier.

A further embodiment of the invention is a method for determining an in vivo cardiac electrophysiology profile of a compound that affects one or more cardiac ion channels via either direct interaction with the cardiac ion channel or secondarily through binding to associated receptors, which comprises administering the compound to a rat, and simultaneously measuring one or more periods selected from the group consisting of an atrial refractory period, an ventricular refractory period, and an AV nodal refractory period, and one or more intervals selected from an electrocardiogram interval and a cardiac electrogram conduction interval.

In another embodiment of the invention, the cardiac ion channel is one that is typically associated with the cardiovascular system, e.g. the potassium, sodium and calcium ion channel.

In another embodiment of the invention, the associated receptor is one associated with cardiac ion channels, e.g., the muscarinic, adenosinergic and serotoninergic receptor.

### DEFINITIONS

An “electrogram”, as used herein, refers to a record on paper or film made by an electrical event. In electrophysiology, an electrogram is a recording taken directly from the surface by unipolar or bipolar leads. A “His electrogram” or “His bundle electrogram is a test that measures electrical activity in a part of the heart known as the bundle of His. The bundle of His is a group of fibers that carry an electrical impulse through the center of the heart to ensure the sequence of the heart's contractions.

An “electrocardiogram” (“ECG”) measures the electrical activity of a heartbeat. Electrical impulses or “waves” traveling through the heart cause the muscle to contract and pump blood. Measurements representing the time required for a wave to travel from one part of the heart to another indicate whether the electrical activity is normal, slow, fast, or irregular.

“MAP” refers to mean arterial pressure.

“HR” refers to heart rate, an indirect measure of sinus node automaticity.

Refractory Periods

Refractory periods are determined using standard paired pacing technique. Briefly, a train of conditioning stimuli at a defined cycle length is delivered followed by an extrastimulus. The coupling interval between the last pulse of the conditioning train and the extrastimulus is then decreased to obtain refractory period.

“Atrial refractory period”, also referred to as “ARP”, is a direct measure of refractoriness of tissue using the pacing/extrastimulus technique. It is the shortest interval between the end of the conditioning train of stimuli and the extra stimulus that permits propagation of the extrastimulus through the atria.

“Ventricular refractory period”, also referred to as “VRP”, is also a direct measure of refractoriness of tissue using the pacing/extrastimulus technique. It is the shortest interval between the end of the conditioning train of stimuli and the extra stimulus that permits propagation of the extrastimulus through the ventricle.

“Atrioventricular nodal refractory period”, also referred to as “AV nodal refractory period” or “AVRP”, is a measure of the ability of the AV node to conduct extrastimulus to the ventricle. It is the shortest interval between the end of the conditioning train of stimuli and the extra stimulus that permits propagation of the extrastimulus through the AV (atrioventricular) node and results in a ventricular depolarization.

“Atrioventricular nodal function”, also referred to as “AV nodal function”, is an assessment of the AV node that can include determination of AV node refractory period and/or determination of AV node conduction, which is assessed by the AH interval.

Measured Intervals

Electrocardiogram and intracardiac conduction intervals are recorded through a computer data acquisition system. Digital calipers are then used to measure multiple electrocardiogram intervals (PR, QRS, QT) and intracardiac conduction parameters (AH and HV intervals).

“Cardiac electrogram conduction intervals” is a general term that relates to measurement of AH and HV intervals.

“AH interval”, also referred to as “AH”, is a measurement acquired from the ventricular electrogram. The AH interval is commonly defined as the distance between the beginning of the atrial depolarization (A) to the beginning of the His bundle electrogram (H). The AH interval represents AV nodal conduction.

“HV interval”, also referred to as “HV”, is a measurement acquired from the ventricular electrogram. The HV interval is commonly defined as the distance between the beginning of the His bundle electrogram (H) to the beginning of the ventricular depolarization. The HV interval represents His-Purkinje conduction

“PR interval”, also referred to as “PR”, is a measurement acquired from the lead II ECG. The PR interval is measured from the beginning of the P wave to the beginning of the QRS complex. The interval is an indirect measure of AV nodal conduction

“QT interval”, also referred to as “QT”, is a measurement acquired from the lead II ECG. The QT interval is measured from the beginning of the QRS complex to the end of the T wave and is an indirect measure of ventricular repolarization. It is often corrected for the heart rate of the subject and is then reported as “QTc”.

“QRS interval”, also referred to as “QRS”, is a measurement acquired from the lead II ECG. The QRS complex is measured from the beginning to the end of the QRS complex and is a measure of ventricular conduction

### EXAMPLE 1

### Assessment of Kv1.5 Antagonists

Adult, male Sprague Dawley rats (240-280 g) were used for all studies. Rats were anesthetized with a mixture of ketamine:xylazine (85 mg/kg:5 mg/kg, ip). Catheters were placed in the left femoral artery and left femoral vein for the measurement of arterial pressure and the administration of test agents, respectively. In addition, a catheter was placed in the right femoral vein to administer a continuous infusion of ketamine:xylazine (45 mg/kg/hr:1.5 mg/kg/hr). A recording/stimulating catheter was then placed in the right jugular vein and advanced to the right atrium. Two electrodes were used to obtain bipolar atrial electrogram recording, and two were used for bipolar pacing of the atria/heart. The right carotid artery was then cannulated with a second recording/stimulating catheter, and the catheter was advance into the left ventricle. Two electrodes were used to obtain bipolar ventricular and His bundle electrograms, and two were used to pace the ventricle. Needle electrodes were placed subcutaneously in the right axillary and left inguinal areas to record lead II electrocardiogram. After equilibration and baseline readings, continuous infusion of test agent or vehicle was started and readings taken at 10 min and 20 min after start of infusion. Blood samples for plasma analysis were obtained from the femoral artery catheter at 10 and 20 min after the start of the infusion. Atrial effective (ARP), ventricular effective (VRP), and AV node effective (AVRP) refractory periods determined as follows: excitation threshold was determined for the atria and ventricles, then a conditioning train of stimuli (S₁, 1 ms duration) at a cycle length of 150 ms was delivered at 1×-2× threshold followed by an extrastimulus (S₂, 1 ms duration) at 2× threshold. The S₁—S₂ coupling interval was decreased by 5 ms, then 1 ms intervals to obtain refractory periods. ARP and VRP were defined as the shortest coupling interval that permitted propagation of an atrial or ventricular extrastimulus. AVRP was defined as the shortest coupling interval that permitted the propagation of the atria extrastimulus through the AV node to elicit ventricular depolarization. In addition, PR, AH (both a measure of AV nodal conduction) and HV (a measure of His-Purkinje conduction) intervals were measured during short (10-20 sec) trains of fixed S₁—S₁ interval pacing of 150 ms. QT interval was obtained during sinus rhythm. Since there is no reported QT rate-correction formula for the rat, and at least one study suggests that QT interval does not change appreciably with changing heart rate in the rat (Hayes E, Pugsley M K, Penz W P, Adaikan G, Walker M J. Relationship between QaT and RR intervals in rats, guinea pigs, rabbits, and primates. J Pharmacol Toxicol Methods. 1994;32:201-7), both the QT interval during sinus rhythm and a rate corrected QT (QTc) using a formula previously validated in mice (QTc=QT/(RR/100)_{1/2} (Mitchell G F, Jeron A, Koren G. Measurement of heart rate and Q-T interval in the conscious mouse. Am J Physiol. 1998;274:H747-51) were reported. Compounds were dissolved in either saline or N,N-dimethylformamide. Intravenous infusion of test agents or vehicles was normalized to body weight.

Two infusion paradigms were tested with two structurally distinct Kv1.5 antagonists, 3-[(dimethylamino)methyl]-6-methoxy-2-methyl-4-phenylisoquinolin-1(2H)-one hydrochloride (described in WO 02/24655 A1) and (2-isopropyl-5-methylcyclohexyl)(diphenyl)phosphine oxide (described in U.S. Pat. No. 6,214,809 B1). In an initial shorter duration infusion paradigm, a 5-min intravenous infusion of compound or vehicle was administered with electrocardiogram/electrophysiology readings determined immediately after the infusion. Briefly, infusion of either 3-[(dimethylamino)methyl]-6-methoxy-2-methyl-4-phenylisoquinolin-1(2H)-one hydrochloride or (2-isopropyl-5-methylcyclohexyl)(diphenyl)phosphine oxide resulted in increases in QT interval, ARP and VRP in a dose dependent manner. Since Kv1.5 has been shown to be expressed in both rat atrium and ventricle, increases in both ARP and VRP were consistent with previously published expression studies (Barry D M, Trimmer J S, Merlie J P, Nerbonne J M. Differential Expression of Voltage-Gated K+ Channel Subunits in Adult Rat Heart: Relation to Functional K+ Channels? Circ Res. 1995;77:361-369). There were no significant changes in vehicle-infused rats. Overall, these data provided evidence that changes in refractory periods and QT intervals following administration of a Kv1.5 antagonist could be demonstrated in the rat. Moreover, these data showed that these effects were dose-dependent, and not unique to a specific structural class of Kv1.5 antagonist.

In an extension of these studies to longer duration infusions, 3-[(dimethylamino)methyl]-6-methoxy-2-methyl-4-phenylisoquinolin-1(2H)-one hydrochloride and (2-isopropyl-5-methylcyclohexyl)(diphenyl)phosphine oxide were tested in a 20-min intravenous continuous infusion protocol. Ten minutes after the start of a 0.1 mg/kg/min 3-[(dimethylamino)methyl]-6-methoxy-2-methyl-4-phenylisoquinolin-1(2H)-one hydrochloride infusion, there was a slight increase in QT (and QTc), but no change in ARP or VRP. However, 20-min after the start of the infusion, there were slight increases in heart rate (5%), and VRP (5%), with larger increases in QT and QTc (10% and 16%, respectively), but no change in ARP. Plasma levels of 3-[(dimethylamino)methyl]-6-methoxy-2-methyl-4-phenylisoquinolin-1(2H)-one hydrochloride at 10 and 20 min after the start of a 0.1 mg/kg/min infusion were 3.4±0.2 μM and 2.8±0.3 μM, respectively.

Increasing the infusion rate of 3-[(dimethylamino)methyl]-6-methoxy-2-methyl-4-phenylisoquinolin-1(2H)-one hydrochloride to 0.2 mg/kg/min resulted in significant increases in VRP and QT 10-min post infusion. Twenty minutes after the start of the 0.2 mg/kg/min infusion, 3-[(dimethylamino)methyl]-6-methoxy-2-methyl-4-phenylisoquinolin-1(2H)-one hydrochloride caused significant increases in heart rate (−17% change in RR interval), ARP (12%), VRP (11%) and QT interval (14%, 24% increase in QTc). Plasma levels of 3-[(dimethylamino)methyl]-6-methoxy-2-methyl-4-phenylisoquinolin-1(2H)-one hydrochloride at 10 and 20 min after the start of the 0.2 mg/kg/min infusion were 6.3±0.5 μM and 5.5±0.2 μM, respectively. Infusion of saline vehicle did not cause significant changes in any of the variables measured.

A limiting factor in primary in vivo screens can be the solubility of test agents in aqueous vehicles. Therefore, we tested the effect of 20-min infusions of DMF on electrocardiogram/electrophysiology parameters, and then compared the electrocardiogram/electrophysiology effects of 3-[(dimethylamino)methyl]-6-methoxy-2-methyl-4-phenylisoquinolin-1(2H)-one hydrochloride dissolved in saline or DMF. Briefly, there were no significant changes in any of the variables measured during a 20-min infusion of 100% DMF. Next, we infused rats with 0.2 mg/kg/min 3-[(dimethylamino)methyl]-6-methoxy-2-methyl-4-phenylisoquinolin-1(2H)-one hydrochloride dissolved in DMF to compare 3-[(dimethylamino)methyl]-6-methoxy-2-methyl-4-phenylisoquinolin-1(2H)-one hydrochloride-dependent electrocardiogram/electrophysiology effects across different vehicles. Overall, both plasma levels and the percent increases in QT, ARP, and VRP were similar in 3-[(dimethylamino)methyl]-6-methoxy-2-methyl-4-phenylisoquinolin-1(2H)-one hydrochloride/saline and 3-[(dimethylamino)methyl]-6-methoxy-2-methyl-4-phenylisoquinolin-1(2H)-one hydrochloride/DMF groups. A 20-min infusion of -[(dimethylamino)methyl]-6-methoxy-2-methyl-4-phenylisoquinolin-1(2H)-one hydrochloride in saline vehicle resulted in 14%, 12%, and 11% increases in QT, ARP, VRP, respectively. Similarly, a 20-min infusion of 3-[(dimethylamino)methyl]-6-methoxy-2-methyl-4-phenylisoquinolin-1(2H)-one hydrochloride in DMF vehicle increased QT, ARP, and VRP, 11%, 8%, and 13%, respectively. In addition to examining the effects of 3-[(dimethylamino)methyl]-6-methoxy-2-methyl-4-phenylisoquinolin-1(2H)-one hydrochloride, we examined the effects of a structurally distinct Kv1.5 blocker, the phosphine oxide (2-isopropyl-5-methylcyclohexyl)(diphenyl)phosphine oxide. Similar to the changes seen during a 0.2 mg/kg/min infusion of 3-[(dimethylamino)methyl]-6-methoxy-2-methyl-4-phenylisoquinolin-1(2H)-one hydrochloride, a 0.2 mg/kg/min infusion of (2-isopropyl-5-methylcyclohexyl)(diphenyl)phosphine oxide caused significant increases in QT, ARP, and VRP 10-min post infusion. Twenty minutes after the start of the infusion, increases in QT (27%, 33% increase in QTc), ARP (24%), and VRP (22%) were larger, but similar, to those observed with 0.2 mg/kg/min 3-[(dimethylamino)methyl]-6-methoxy-2-methyl-4-phenylisoquinolin-1(2H)-one hydrochloride.

Table 1 shows data corresponding to Example 1.

### EXAMPLE 2

### Assessment of hERG Antagonists

Using the method described in example one, we tested the effect of hERG antagonists in the rat electrocardiogram/Electrophysiologic model. The rat equivalent of hERG, rERG, has been reported to be expressed in rat atria and ventricle (Pond A L, Scheve B K, Benedict A T, Petrecca K, Van Wagoner D R, Shrier A, Nerbonne J M. Expression of distinct ERG proteins in rat, mouse, and human heart. Relation to functional I(Kr) channels. J Biol Chem. 2000;275:5997-6006, and Wymore R S, Gintant G A, Wymore R T, Dixon J E, McKinnon D, Cohen I S. Tissue and species distribution of mRNA for the IKr-like K+ channel, erg. Circ Res. 1997;80:261-8). Furthermore, IKr current has been observed in rat cardiac myocytes, and blockade of this channel with well-known IKr blockers decreased potassium current in vitro (Pond A L, Scheve B K, Benedict A T, Petrecca K, Van Wagoner D R, Shrier A, Nerbonne J M. Expression of distinct ERG proteins in rat, mouse, and human heart. Relation to functional I(Kr) channels. J Biol Chem. 2000;275:5997-6006, and Wymore R S, Gintant G A, Wymore R T, Dixon J E, McKinnon D, Cohen I S. Tissue and species distribution of mRNA for the IKr-like K+ channel, erg. Circ Res. 1997;80:261-8). To determine whether blocking IKr had effects on the electrocardiogram/electrophysiology profile in rats, a 20-min continuous infusion of the IKr blocker, N-[1′-(6-cyano-1,2,3,4-tetrahydro-2(R)-naphthalenyl)-3,4-dihydro-4(R)-hydroxyspiro[2H-1-benzopyran-2,4′-piperidin]-6-YL]-,(+)-,monohydrochloride (described in U.S. Pat. No. 5,206,240) was administered to rats. Infusion of N-[1′-(6-cyano-1,2,3,4-tetrahydro-2(R)-naphthalenyl)-3,4-dihydro-4(R)-hydroxyspiro [2H-1-benzopyran-2,4′-piperidin]-6-YL]-,(+)-,monohydrochloride at a dose that produced a similar increase in VRP as the Kv1.5 antagonists, resulted in a marked increase in AVRP (21%) and a marked delay in AV node conduction (˜13% increase in AH interval) a plasma concentrations of ˜600 nM. In addition, increases in MAP (18%) and HR (22%) were also noted. Infusion of lower doses of N-[1′-(6-cyano-1,2,3,4-tetrahydro-2(R)-naphthalenyl)-3,4-dihydro-4(R)-hydroxyspiro[2H-1-benzopyran-2,4′-piperidin]-6-YL]-,(+)-,monohydrochloride resulted in significant changes in AVRP with no change in ARP or VRP and at plasma concentration of 27 nM. These data demonstrate that this model is useful for assessment of in vivo IKr activity.

Table 2 shows data corresponding to Example 2.

TABLE 1
Percent change ± se after a 20 min infusion of either 3-[(dimethylamino)methyl]-6-methoxy-2-methyl-4-
phenylisoquinolin-1(2H)-one hydrochloride (compound 1) or (2-isopropyl-5-methylcyclohexyl)
(diphenyl)phosphine oxide (compound 2).

| Agent | mg/kg/min | MAP | HR | QT | QTc | VRP | ARP | AVRP | PR | QRS | AH | HV |
|---|---|---|---|---|---|---|---|---|---|---|---|---|
| Saline | 0.1 | 3 ± 2 | 4 ± 2 | 1 ± 2 | 3 ± 3 | −2 ± 4 | 1 ± 1 | 1 ± 1 | 0 ± 1 | 1 ± 2 | 2 ± 1 | −2 ± 4 |
| DMF | 0.04 | 0 ± 3 | 3 ± 3 | −1 ± 1 | 0 ± 2 | −2 ± 1 | −2 ± 2 | −1 ± 1 | −1 ± 1 | 0 ± 2 | −1 ± 2 | −2 ± 2 |
| compound 1 | 0.2 | 2 ± 2 | −1 ± 4 | 12 ± 2 | 11 ± 2 | 13 ± 1 | 5 ± 3 | 4 ± 2 | 3 ± 1 | 0 ± 0 | 5 ± 1 | 0 ± 3 |
| compound 2 | 0.2 | 4 ± 4 | 8 ± 6 | 27 ± 7 | 33 ± 8 | 22 ± 5 | 26 ± 4 | −1 ± 1 | −3 ± 1 | −3 ± 4 | −1 ± 1 | −8 ± 3 |

TABLE 2
Percent change ± se after a 20 min infusion of N-[1′-(6-cyano-1,2,3,4-tetrahydro-2(R)-naphthalenyl)-3,4-
dihydro-4(R)-hydroxyspiro[2H-1-benzopyran-2,4′-piperidin]-6-YL]-,(+)-, monohydrochloride (Ikr blocker).

| Test Agent | mg/kg/min | MAP | HR | QT | QTc | VRP | ARP | AVRP | PR | QRS | AH | HV |
|---|---|---|---|---|---|---|---|---|---|---|---|---|
| Ikr blocker | 0.0001 | −7 ± 8 | −1 ± 5 | 1 ± 1 | 0 ± 2 | −2 ± 1 | 0 ± 2 | 1 ± 2 | 2 ± 2 | 0 ± 1 | 2 ± 1 | −1 ± 1 |
| | 0.001 | −1 ± 3 | 3 ± 4 | −1 ± 1 | 1 ± 1 | 0 ± 1 | 2 ± 4 | 11 ± 1 | 8 ± 1 | 3 ± 1 | 9 ± 1 | −4 ± 3 |
| | 0.03 | 18 ± 5 | 22 ± 5 | 11 ± 3 | 23 ± 5 | 7 ± 2 | 4 ± 4 | 21 ± 3 | 9 ± 4 | 0 ± 1 | 13 ± 4 | 4 ± 3 |

### EXAMPLE 3

### Assessment of Calcium Channel Antagonists

Using the model described in example one, we assessed whether calcium channel antagonists would have similar effect on cardiac electrocardiogram and electrophysiology in rats as compared to published literature in large animals and humans. To this end, we infused amlodipine or diltiazem to provide assessment of two structurally distinct calcium channel blockers that have different cardiac electrophysiologic effects. Among the differences between amlodipine and diltiazem clinically, is the fact the diltiazem had more pronounced effects on AV nodal function than amlodipine.

Infusion of 0.03 mg/kg/min amlodipine for 20 min resulted in a modest decrease in blood pressure (−14%) and no significant change in heart rate. There was no change in AV nodal conduction or AV node refractoriness. In contrast, infusion of diltiazem at a dose that caused a similar decrease in blood pressure resulted in a significant decrease in heart rate (12%) and significant increases in AV nodal conduction (9%), and AV nodal refractoriness (10%).

At higher infusion rates of amlodipine that caused drastic decreases in blood pressure (30%), there were only slight increases in AV nodal conduction (6%) and AV nodal refractoriness (7%). In contrast, doses of diltiazem that caused similar blood pressure lowering (23%) caused greater increases in AV nodal conduction (15%), and AV nodal refractoriness (19%). None of the experiments using calcium channel antagonists caused significant increase in ventricular refractoriness.

### EXAMPLE 4

### Assessment of Sodium Channel Antagonists

Using the model described in example one, we assessed whether infusion of a sodium channel blocker in this rat model would result in characteristic changes in conduction that occur in large animals model and in human. Infusion of either procainamide (Class 1a) or propafenone (Class 1c) at increasing rates resulted in dose-dependent increases in QRS interval and HV interval without having significant effects on blood pressure or heart rate.

Overall, these data demonstrate the feasibility of measuring basal and compound-dependent changes in electrocardiogram intervals, cardiac conduction intervals, and atrial, AV node, and ventricular refractory periods in a rat model. The described rat electrocardiogram/electrophysiology model is therefore useful as an assay for assessing compound dependent effects on cardiac electrophysiology.

What is claimed is:

1. A method for determining an in vivo cardiac electrophysiology profile of a compound affecting one or more cardiac ion channels which comprises administering the compound to a rat, and simultaneously measuring one or more periods selected from the group consisting of the atrial refractory period, the ventricular refractory period, and the AV nodal refractory period, and one or more intervals selected from an electrocardiogram interval and a cardiac electrogram conduction interval.

2. A method claim 1, which comprises
1) cannulating the left femoral artery of the rat with a first catheter,
2) cannulating the left femoral vein of the rat with a second catheter and the right femoral vein of the rat with a third catheter,
3) introducing a first recording and stimulating catheter into the right jugular vein of the rat, and advancing the first recording and stimulating catheter into or near the right atrium of the rat to pace the heart of the rat,
4) advancing a second recording and stimulating catheter down the right common carotid of the rat into the left ventricle of the rat,
5) placing needle electrodes subcutaneously at the right axillary and left inguinal areas of the rat,
6) administering the test compound either continuously or intermittently intravenously, and
7) determining one or more intervals selected from the group consisting of an electrocardiogram interval and a cardiac electrogram conduction interval and one or more periods selected from the group consisting of an atrial refractory period, an ventricular refractory period, and an AV nodal refractory period.

3. A method of claim 1, where the test compound is a Kv1.5 antagonist.

4. A method of claim 1, where the cardiac ion channel is the Kv1.5 potassium ion channel.

5. A method of claim 1, where the test compound is a sodium channel antagonist.

6. A method of claim 1, where the test compound is a calcium channel antagonist.

7. A method of claim 1, where the test compound is an ERG potassium channel inhibitor.

8. A method of claim 1, where the test compound is a cardiac refractoriness modifier or a cardiac conduction modifier.

9. A method for determining an in vivo cardiac electrophysiology profile of a compound that affects one or more cardiac ion channels via either direct interaction with the cardiac ion channel or secondarily through binding to an associated receptor, which comprises administering the compound to a rat, and simultaneously measuring one or more periods selected from the group consisting of an atrial refractory period, a ventricular refractory period, and an AV nodal refractory period, and one or more intervals selected from an electrocardiogram interval and a cardiac electrogram conduction interval.

10. A method of claim 9, wherein the cardiac ion channel is selected from the group consisting of the potassium ion channel, the sodium ion channel, and the calcium ion channel.

11. A method of claim 10, wherein the associated receptor is selected from the group consisting of the muscarinic receptor, the adenosinergic receptor and the serotoninergic receptor.
